(19) United States
(12) Patent Application Publication
(10) Pub. No.: US 2025/0384965 A1
(43) Pub. Date: Dec. 18, 2025

(54) METHOD FOR DETERMINING THE COMPOSITION OF A REACTION SYSTEM

(71) Applicant: Covestro Deutschland AG, Leverkusen (DE)

(72) Inventors: Christoph Loschen, Haan (DE); Jakob Schneider, Elsdorf (DE); Anja Mackenzie, Münster (DE); Jan Meyer, Wuppertal (DE)

(21) Appl. No.: 18/877,714

(22) PCT Filed: Jun. 21, 2023
(86) PCT No.: PCT/EP2023/066882

§ 371 Date: Dec. 20, 2024

(30) Foreign Application Priority Data
Jun. 29, 2022 (EP) 22182060.8

(51) Int. Cl. G16C 20/10

(52) U.S. Cl. CPC G16C 20/10

(57) ABSTRACT

A computer-implemented method which comprises the automated and rational (computer aided) modification of chemical recipes, in particular the design of the concrete starting composition of a reaction system, for example an oligomeric or polymeric polyol system, using an optimization procedure aiming at maximizing the similarity to an original target/reference system is provided. Furthermore, a data processing apparatus comprising means for carrying out the method, a computer program product comprising instructions which, when the program is executed by a computer, cause the computer to carry out the method and a computer-readable storage medium comprising instructions which, when executed by a computer, cause the computer to carry out the method is provided.

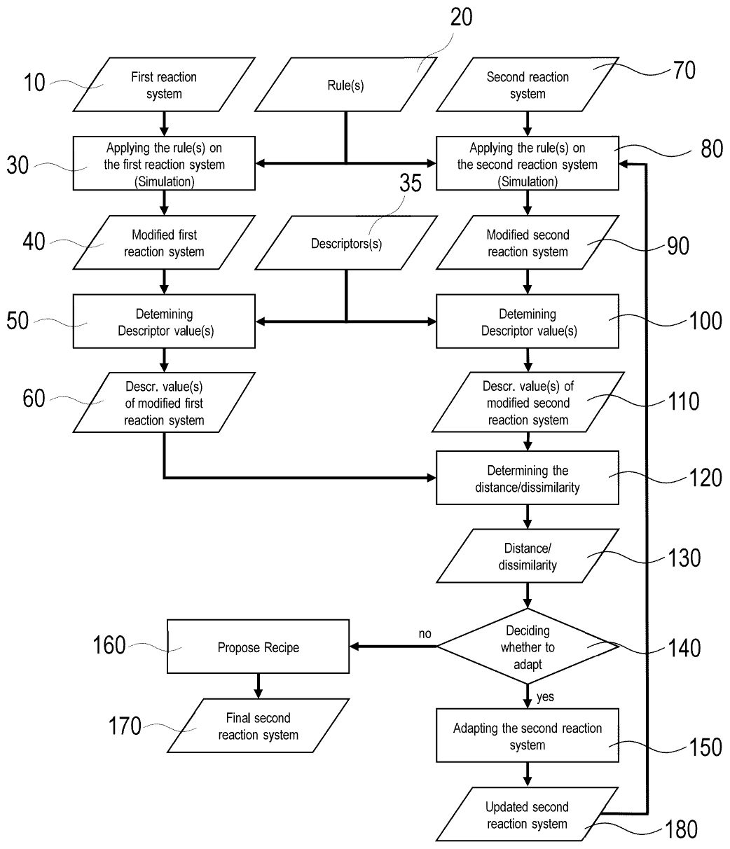

### CROSS REFERENCE TO RELATED APPLICATIONS

This application is a U.S. national stage application, filed under 35 U.S.C. § 371, of International Application No. PCT/EP2023/066882, which was filed on Jun. 21, 2023, and which claims priority to European Patent Application No. 22182060.8, which was filed on Jun. 29, 2022. The entire contents of each are hereby incorporated by reference into this specification.

### FIELD

The present invention relates to a computer-implemented method for designing the starting composition of a chemical reaction system for maximizing the similarity to a reference system in order to substitute individual components in the reference reaction system. The invention further relates to a data processing apparatus comprising means for carrying out the method, a computer program product comprising instructions which, when the program is executed by a computer, cause the computer to carry out the method and a computer-readable storage medium comprising instructions which, when executed by a computer, cause the computer to carry out the method.

### BACKGROUND

The computer-aided modification of chemical information using an optimization procedure, in particular the design of a specific starting material composition for a reaction system, is the subject of ongoing efforts. In order to further reduce the dependency on raw materials based on petrochemistry and to comply with tightened regulatory requirements it is of particular interest to use optimizations procedures in order to substitute those ingredients in reaction systems while changing the material properties of the later product as little as possible.

US 2020/342960 A1 discloses a target-based drug screening method using inverse quantitative structure-(drug) performance relationships (QSPR) analysis and molecular dynamics simulation. The method includes modeling a molecular structure of a test compound group against a target molecule, obtaining a quantitative structure-(drug) performance relationships (QSPR) of the test compound group, acquiring the optimal pharmacophore of a novel target-based drug through a numerical inversion of the QSPR, and selecting drug candidates having a molecular structure similar to the optimum pharmacophore from the test compound group. The approach is restricted to single molecules and not suited for reaction mixtures or polymers. The optimization is targeted directly towards experimental properties, that have either a high level of uncertainty when being predicted computationally or being scarcely available. Maranas et al. address the design of polymers with optimal levels of macroscopic properties through the use of topological indices. Specifically, two zeroth-order and two first-order connectivity indices are for the first time employed as descriptors in structure-property correlations in an optimization study. Based on these descriptors, a set of new correlations for heat capacity, cohesive energy, glass transition temperature, refractive index, and dielectric constant are proposed. These correlations are incorporated into an optimization framework. (Camarda, Kyle V., and Costas D. Maranas. “Optimization in polymer design using connectivity indices.” Industrial & Engineering Chemistry Research 38.5 (1999): 1884-1892.) This approach does not take into account the intrinsic polydisperse nature of polymers, i.e. is restricted to ideal polymers modelled as infinite chains. Hence, no concrete recipes can be proposed by these approach. The optimization is targeted directly towards experimental properties, that have either a high level of uncertainty when being predicted computationally or being scarcely available.

Faeder et al. describe rule-based modeling with BioNetGen to study the dynamics of complex biochemical systems. Rule-based models can be analyzed by carrying out deterministic or stochastic simulations. However, they are often difficult to calibrate due to many unknown parameters and limited experimental training data. They present a generic parameter estimation framework for calibrating rule-based models. The experimental data as well as qualitative properties of the system are encoded as a specification formula in a bounded linear temporal logic. Given a candidate set of parameter values they apply the statistical model checking procedure to evaluate the quality of this candidate set. Based on the outcome, a new set of parameters is chosen using a standard global search strategy. (Liu, Bing, and James R. Faeder. “Parameter estimation of rule-based models using statistical model checking.” 2016 IEEE International Conference on Bioinformatics and Biomedicine (BIBM). IEEE, 2016.) This approach is limited to biological systems, i.e. not suited for polymer recipes. Furthermore is does not use an atomistic resolution of the species and hence is limited concerning the computation of detailed descriptors.

Daoutidis et al. have proposed a strategy that simultaneously identifies (a) the most desirable biomass-derived products for an application of interest and (b) the corresponding synthesis routes. The strategy consists of i) constructing an exhaustive network of reactions consistent with an input set of chemistry rules and ii) using the network information to formulate and solve an optimization problem that yields an optimal product distribution and the sequence of reactions that synthesize them. They use this strategy to identify potential renewable oxygenates and hydrocarbons obtained from heterogeneous catalysis of biomass that can be blended with gasoline to satisfy ASTM specifications. Multiple objectives (energy loss, catalyst requirement, and absolute heat duty) are considered, and multiple alternative solutions are found in each case (Marvin, W. Alex, Srinivas Rangarajan, and Prodromos Daoutidis. “Automated generation and optimal selection of biofuel-gasoline blends and their synthesis routes.” Energy & Fuels 27.6 (2013): 3585-3594.) The approach is restricted to single molecules and not suited for realistic reaction mixtures or polymers. The optimization is targeted directly towards experimental properties, that have either a high level of uncertainty when being predicted computationally or being available only for small datasets. Mavrantzas et al. report a multi-scale model-based approach that combines a computer aided molecular design technique based on group contribution models for predicting polymer repeat unit properties with atomistic simulations for providing first-principles arrangements of the repeat units and for predictions of physical properties of the chosen candidate polymer structures. The method has been developed and tested for design of polymers with desired properties (Satyanarayana, Kavitha Chelakara, et al. “Computer aided polymer design using multi-scale modelling.” Brazilian Journal of Chemical Engineering 27 (2010): 369-380.) The approach is restricted to polymers modelled as infinite chain of repeat units, not dedicated to realistic reaction mixtures or polydisperse polymers.

So far, no method has been described that optimizes a complex reaction system with respect to its starting composition, i.e. proposing concrete alternative recipes, using the similarity of a set of descriptors derived from its components at a monomeric and atomistic resolution.

Computer aided design methods in the art as mentioned above are usually based on computer-predicted properties that are used to define a desired target value or range for mostly well-defined single compounds or for polymers which are modelled as a monodisperse system of infinite chain length. In practice, those methods are hampered by several issues. First, the prediction of molecular properties with chemical accuracy for a molecule, a molecular mixtures or a polymer is still an unsolved challenge for many chemical systems and usually any property prediction is associated with a some inaccuracy, when compared to the experiment, that may deteriorate any subsequent optimization procedure. Secondly, the systems are always assumed to be of a monodisperse character, but for many systems, in particular oligomeric and polymeric ones, the polydisperse character is important, as for example different molecular chain lengths and/or the effect of end groups is non-negligible. In fact, most chemical systems are inherently mixtures with a complex composition instead of pure well-defined systems.

### SUMMARY

The present invention has the object of at least partially overcoming the drawbacks in the art. In particular, the invention has the objective of efficiently and accurately designing chemical reaction systems with the aim to substitute certain raw materials, e.g. by biobased materials, i.e. single or multiple ingredients in a chemical reference reaction system while largely maintaining the material properties of the product produced from the chemical reaction system.

This object is achieved by a method according to claim 1. A data processing apparatus is the subject of claim 13, a computer program product the subject of claim 14 and a computer-readable storage medium the subject of claim 15. Preferred embodiments are the subject of the dependent claims. They may be combined freely unless the context clearly indicates otherwise.

### BRIEF DESCRIPTION OF THE DRAWING

The FIGURE shows a flow chart of a method according to the present disclosure.

### DETAILED DESCRIPTION

Accordingly, a computer-implemented method for designing the starting composition of a chemical reaction system for maximizing the similarity to a reference reaction system in order to substitute individual components in the reference system comprises:
A) Providing information on a reference reaction system, the information comprising the components of the reference reaction system and their relative amounts in the reference reaction system;
B) Providing information on a test reaction system, the information comprising the components of the test reaction system and their relative amounts in the test reaction system;
C) Providing at least one first rule for forming and/or cleaving chemical bonds from the components of the reference reaction system and/or species formed from components of the reference reaction system;
D) Applying the at least one rule on the reference reaction system, thereby obtaining a modified reference reaction system;
E) Providing at least one descriptor for at least part of the modified reference reaction system and for at least part of a modified test reaction system as recited in step H);
F) Determining descriptor value(s) from the at least one descriptor for at least part of the modified reference reaction system;
G) Providing at least one second rule for forming and/or cleaving chemical bonds from the components of the test reaction system and/or species formed from components of the test reaction system;
H) Applying the at least one second rule on the test reaction system, thereby obtaining a modified test reaction system;
I) Determining descriptor value(s) from the at least one descriptor as recited in step E) for at least part of the modified test reaction system;
J) Determining the distance and/or the dissimilarity between the values of the at least one descriptor from steps F) and I) using a measure for the distance in a descriptor space occupied by the descriptors;
K) Deciding to adapt or not to adapt the test reaction system depending on the result of the determination of the distance and/or dissimilarity in step J);
L) If the decision to adapt in step K) has been taken, adapting the test reaction system by changing the components of and/or their relative amounts in the test reaction system yielding an updated test reaction system and subsequently repeating the method starting at step H) based on the updated test reaction system.

The method according to the example has several advantages. The method computes a descriptor-based similarity between a first chemical reaction system, i.e. the reference reaction system, and a new (2nd reaction system) system, i.e. the test reaction system. The method is not predicting just target properties for a new system. This avoids some problems due to inaccurately computed properties or insufficient models, which typically occur for complex chemical systems. In other words, not a reference range of properties is specified as objective for the optimization problem, but a reference reaction system which undergoes the same descriptor computation as the chemical system to be optimized, i.e. the test reaction system. The direct prediction of (target) properties is avoided and the overall optimization scheme is carried out using the concept of descriptor similarity. The underlying idea is to benefit from error cancellation, as the descriptors are computed for both systems involving the same approximations and assumptions in both cases.

Another advantage of the method is that it takes explicitly into account the polydisperse character of an oligomeric or polymeric reaction system, by using a kinetic model as basis for any subsequent computations, whereas so far only monodisperse pure and well-defined systems have been used. In addition, a complete recipe, i.e. a composition of a reaction mixture can be proposed with the proposed invention, whereas so far only pure and non-reactive single molecular structures are the targets of computer aided design.

Altogether, this approach allows for the rapid computer aided proposition of new chemical recipes of reactive systems that are most similar to a given reference system. This is particular useful for the efficient substitution of raw materials, e.g. by biobased materials, i.e. single or multiple ingredients of a chemical recipe, which have to be exchanged for example due to regulative or economic reasons, without compromising on the overall product's material properties. As the properties of the product created from the proposed recipe, i.e. the test reaction system, will already be very close to the reference reaction system, much less experimental iterations are needed in order to adopt the recipe according to its changed ingredients.

Computer-based methods similarity searching of chemical reactions are generally known from the art. For example, US 2009/024575 A1 discloses a computer based method for similarity searching of chemical reactions in order to investigate large reaction databases. Here, a query chemical reaction is characterized by structural descriptors and compared via their similarity/distance to reaction descriptors stored in a reaction database. As opposed to the present invention, where the chemical reactions of the reference and the test system including their rate constants are already defined at the beginning of the workflow, no relation is made to any concrete reaction reactant or product, neither mono-nor polydisperse, nor to any kinetics e.g. in the form of chemical rate constant. Only the abstract chemical transformation itself, of molecules into each other, is subject to a similarity search. Hence, no reaction products are obtained nor any recipe can be proposed with this approach.

According to the invention information on a reference reaction system (step A)) and information on a test reaction system is provided (step B)). Both the reference and the test reaction systems may possess a large variety of chemical components. According to the first embodiment of the invention the components of the reference reaction system as recited in step A) and/or the components of the test reaction system as recited in step B) comprise alkylene oxides, polyfunctional carboxylic acids, carboxylic acid anhydrides, cyclic ethers, carbon dioxide, cyclic carbonates, polyols, polyisocyanates, polyamines, aromatic hydrocarbons, olefins, (meth) acrylates, bisphenols, phosgene, dialkyl carbonates, aldehydes, lactames, glycolides, amino acids, hydroxy-substituted carboxylic acids, or a combination of at least two of the aforementioned components.

According to step C) of the computer-implemented method according to the invention at least one first rule for forming and/or cleaving chemical bonds from the components of the reference reaction system and/or species formed from components of the reference reaction system is provided. Moreover, according to step G) at least one second rule for forming and/or cleaving chemical bonds from the components of the test reaction system and/or species formed from components of the test reaction system is provided.

According to an embodiment of the invention the at least one first rule as recited in C) comprises which functional group of a component is present, which functional group of a component is available for forming a covalent bond or for cleaving a covalent bond, which functional groups of components can react with other functional groups, a threshold criterion when the forming or the cleavage of a covalent bond occurs or a combination of at least two of the aforementioned rules.

Preferably the at least one first rule of step C) is identical to the at least one second rule of step G). This ensures that the reference reaction system and the test reaction system are modified according to similar chemical transformations.

In an preferable embodiment relative rate constants which are derived and/or measured from kinetic experiments may be used as input data for setting up the at least one first rule of step C) and/or the at least one second rule reaction rules of step G). In alternative, these rules may be simulated. However, the simulated may be not accurate enough in certain instances.

According to step D) of the method according to the invention the at least one first rule is applied on the reference reaction system, thereby obtaining a modified reference reaction system. According to a preferred embodiment applying the at least one first rule as recited in step D) comprises running a kinetic Monte Carlo-simulation, running a molecular Monte Carlo-simulation, running a molecular dynamics simulation, running a Miller-Macosko type calculation. Here, especially a kinetic Monte-Carlo simulation proves to be very efficient and fast compared to other types of simulations.

Preferably the modified reference reaction system obtained by applying the at least one first rule on the reference reaction system comprises an ensemble of discrete components and information on the discrete components of the modified reference reaction system and their relative amounts in the modified reference reaction system.

According to step E) of the method according to the invention at least one descriptor for at least part of the modified reference reaction system and for at least part of a modified test reaction system as recited in step H) is provided. In other words: according to the invention it is ensured that identical descriptors are used both for the modified reference reaction system and for the modified test reaction system. This in turn ensures that a valid distance/similarity metric according to Step J) can be determined.

The at least one descriptor may be selected from a very large variety of descriptors. According to an embodiment of the invention the at least one descriptor as recited in step E) is a statistical/compositional descriptors, preferably number average molecular weight, weight average molecular weight, viscosity average molecular weight, the polydispersity index, the mean functionality, the OH number, the acid number.

It is also possible that the at least one descriptor is a topological descriptor, preferably Wiener Index, Randic connectivity index, Balaban-J index, Ipc index, Zagreb index, Bertz index, chi molecular connectivity indices, Kier-Hall valence connectivity index, seniority and priority indices, kappa shape indices, BCUT indices, Estate indices, Walk and path counts, extended connectivity fingerprints.

It is further possible that the at least one descriptor is a molecular descriptor, preferably Labute's approximate surface area, solvent-accessible surface area, radius of gyration, 2D autocorrelation, Eigenvalue-based descriptors, RDF descriptors, 3D-MORSE descriptors, WHIM descriptors, GETAWAY descriptors, functional group counts, number of rotatable bonds, number of hydrogen acceptors and donors, Gasteiger/Marsili Partial Charges, topological polar surface area, molar refractivity.

It is further possible that the at least one descriptor is a force field based descriptors, preferably intramolecular potential energy, radial distribution function, bond angle distribution function, dihedral distribution functions; and/or a quantum chemical descriptor, preferably atomic charges, HOMO and LUMO energies, molecular hardness, molecular polarizability, dipole moment, total energy, molecular quantum number descriptor.

It is further possible that the at least one descriptor is a thermodynamic descriptor, preferably octanol-water partition coefficient, sigma-profile, sigma moment, polarity moment vector, activity coefficient, chemical potential, free energy, octanol-water partition coefficient, solubility.

It is further possible that the at least one descriptor is a health, sustainability or environment related descriptor, preferably toxicity, carbon dioxide equivalents, carbon footprint, energy consumption, product recycling rate, sustainability index, supply chain miles, water footprint, price.

Preferably, a combination of at least two of the aforementioned descriptors is selected.

Using the at least one descriptor thus provided it is possible to determine one or more descriptor values from the at least one descriptor for at least part of the modified reference reaction system as taught by step F) of the method according to the invention.

If the modified reference reaction system comprises an ensemble of discrete components and information on the discrete components of the modified first reaction system and their relative amounts in the modified reference reaction system, as mentioned above, it is preferred that the determination of the descriptor value(s) from the at least one descriptor for at least part of the modified reference reaction system in step F) is carried out using atomistic resolution information for each discrete component. In case the at least one descriptor is a thermodynamic descriptor, as mentioned above, those descriptors preferably contain information concerning polarity and solubility, and are preferably obtained from the liquid phase thermodynamics approach COSMO-RS. Here, the previously obtained atomistic resolution information is used to construct the molecular input (σ-profiles) for the COSMO-RS calculation and descriptor generation

According to step G) of the method according to the invention at least one second rule for forming and/or cleaving chemical bonds from the components of the test reaction system and/or species formed from components of the test reaction system is provided.

According to a preferred embodiment of the invention the at least one second rule—as in case of the at least one first rule recited in step C)—comprises which functional group of a component is present, which functional group of a component is available for forming a covalent bond or for cleaving a covalent bond, which functional groups of components can react with other functional groups, a threshold criterion when the forming or the cleavage of a covalent bond occurs or a combination of at least two of the aforementioned rules.

As mentioned above it is possible that the at least one second rule may be identical to the at least one first rule.

According to step H) of the method according to the invention the at least one second rule is applied on the test reaction system, thereby obtaining a modified test reaction system. It is preferred that the at least one second rule again comprises running a kinetic Monte Carlo-simulation, running a molecular Monte Carlo-simulation, running a molecular dynamics simulation, running a Miller-Macosko type calculation.

Preferably the modified test reaction system obtained by applying the at least one second rule on the test reaction system, i.e. the system to be optimized—as in case of the modified reference reaction system-comprises an ensemble of discrete components and information on the discrete components of the modified test reaction system and their relative amounts in the modified test reaction system.

In step I) of the method according to the invention one or more descriptor values are determined from the at least one descriptor as recited in step E) for at least part of the modified test reaction system.

Again, if the modified test reaction system comprises an ensemble of discrete components and information on the discrete components of the modified test reaction system and their relative amounts in the modified test reaction system, as mentioned above, it is preferred that the determination of the descriptor value(s) from the at least one descriptor for at least part of the modified test reaction system in step I) is carried out using atomistic resolution information for each discrete component.

With the descriptor value(s) for both the modified reference reaction system and the modified test reaction system being determined according to steps F) and I) it is possible to determine (step J)) the distance and/or the dissimilarity between the values of the at least one descriptor from steps F) and I) using a measure for the distance in a descriptor space occupied by the descriptors.

In a preferred embodiment the distance and/or dissimilarity as recited in step J) comprises the euclidean/L2 distance, the manhattan/L1/cityblock distance, Canberra distance, Chebyshev distance, Mahalanobis distance, Minkowski distance, Rogers-Tanimoto dissimilarity, Russell-Rao dissimilarity, Sokal-Michener dissimilarity, Sokal-Sneath, mean absolute percentage error, Yule dissimilarity, cosine dissimilarity, dice dissimilarity or a combination of at least one of the aforementioned distances and/or dissimilarities.

Depending on the result of the determination of the distance and/or dissimilarity in step J) it is decided whether to adapt or not to adapt the test reaction system. Many different decision criteria may be applied. According to preferred embodiment the decision as recited in step K) comprises one or more of the following a decision criteria: a threshold value for the distance and/or dissimilarity in step J); a threshold value for the change in distance and/or dissimilarity relative to one or more previously executed steps J); a threshold value for the execution time for the method; a threshold value for the number of repetitions of the method; a threshold value for at least one of the descriptors. Here, the application of a threshold value for the change in distance and/or dissimilarity relative to one or more previously executed steps J) is preferred.

If the decision to adapt the test reaction system, i.e. the system to be optimized, in step K) has been taken the test reaction system is adapted in step L) of the method by changing the components of and/or their relative amounts in the test reaction system thus yielding an updated test reaction system.

Preferably, the adaptation of the test reaction system as recited in step L) is carried out by executing an algorithm selected from: random search, grid search, Bayesian optimization, simplex optimization, evolutionary optimization, genetic algorithm, particle swarm optimization, Metropolis-Hastings Markov-chain Monte-Carlo, adaptive Markov-chain Monte-Carlo, simulated annealing, parallel tempering, mixed linear and non-linear programming or a combination of at least two of the aforementioned algorithms.

Using the adapted test reaction system the method according to the invention is repeated starting at step H), i.e. by subsequently repeating the method starting at step H) based on the updated test reaction system. This means that the at least one second rule is applied also on the updated test reaction system, thereby again obtaining a modified (updated) test reaction system. Subsequently, the at least one descriptor as provided in step E) of the method according to the invention is used to again determine one or more descriptor values for at least part of the modified (updated) test reaction system.

This one or more descriptor value for the modified (updated) test reaction system is then used to again determine the distance and/or the dissimilarity between this/these value(s) and the value(s) obtained for the modified reference reaction system in step F).

If the adaption of the test reaction system in step L) has been carried out in a suitable way the distance/dissimilarity determined one more time in step J) should be reduced. This iteration of steps H) to L) with an updated test reaction system may be performed until the distance/dissimilarity drops below a value that is satisfactory for the user.

If, normally after a certain number of iterations, the value for the distance and/or the dissimilarity between the descriptor value(s) of the modified reference reaction system and the descriptor value(s) drops below a certain threshold value it is decided in step K) to no longer adapt the test reaction system, i.e. the optimization of the test reaction system is completed. In this case an optimized or final test reaction system is proposed and preferably communicated to a user. It can be communicated by all suitable means to the user, e.g. by a personal computer or a mobile device. The user may then use the optimized or final test system as a starting composition of a chemical reaction to produce certain chemicals, in particular with certain raw materials (traditionally based on petrochemistry) substituted by biobased materials in the course of the green transformation of the chemical industry.

Another aspect of the present invention relates to a data processing apparatus comprising means for carrying out the method of one of claims 1 to 12.

A further aspect of the present invention relates to a computer program product comprising instructions which, when the program is executed by a computer, cause the computer to carry out the method of one of claims 1 to 12.

An even further aspect of the present invention relates to a computer-readable storage medium comprising instructions which, when executed by a computer, cause the computer to carry out the method of one of claims 1 to 12.

The present invention will be further described with reference to the FIGURE without wishing to be limited by the FIGURE.

In connection with the FIGURE, an example workflow will be described in detail.

The following example workflows are carried out as follows, with the aim to substitute raw materials within a polymer recipe (the test reaction system), for example due to regulatory or economic reasons, without compromising on its properties in a subsequent application. The workflow is fully automated and coded in Python.

### Example 1

The example workflow is carried out as follows, with the aim to substitute starter molecules for a polyol recipe (the test reaction system) without compromising on its properties in a subsequent application e.g. in a polyurethane reaction. The workflow is fully automated and coded in Python.

A reference reaction system 10 (the first reaction system) is defined to consists of the following chemical compounds, i.e. recipe: ethylene oxide (0.6), glycerol (0.2) and ethylene glycol (0.2) with the composition in mass fractions in parenthesis. Glycerol is defined to have three alcohol groups, ethylene glycol two alcohol groups and ethylene oxide one reactive epoxide group.

A kinetic Monte Carlo simulation (as for example in Suderman, Ryan, et al. “Generalizing Gillespie's direct method to enable network-free simulations.” Bulletin of mathematical biology 81.8 (2019): 2822-2848.) is set up where a number of kinetic rules 20 are defined to allow to react the alcohol groups with epoxide groups with a relative rate constant of 1, i.e. the functional groups of the identified components have the same reactivity. The kinetic rules 20 are applied on the first reaction system 10 (step 30) by carrying out the simulation using 100.000 initial seed molecules. A discrete representation of a polymer is obtained (the modified first reaction system 40), after all reactive groups have been reacted.

Subsequently a number of descriptors 35 is selected and corresponding descriptor values 60 are computed (step 50) from this modified reference reaction system 40 as shown in Table 1: The selected descriptors 35 are: Polydispersity (PD), the OH number (OHZ), the average number of OH groups per molecule (fn(OH)), the topological polar surface area (TPSA) and the Wiener index as obtained from the molecular graph. Explanations regarding the Wiener Index can be found in Todeschini, Roberto, and Viviana Consonni. “Handbook of molecular descriptors”, John Wiley & Sons, 2008. The descriptors values 60 are saved for later usage.

A new trial system (test reaction system 70) is then defined, using starters different from the reference reaction system in the present case ethylene oxide, trimethylolpropane and 1,2-propylene glycol presently using the same set of kinetic rules 20 as defined for the reaction system 10. This trial system is the system to be optimized in the present method. Trimethylolpropane is defined to have three alcohol groups, 1,2-propylene glycol two alcohol groups and ethylene oxide one reactive epoxide group. The composition in mass fractions is initiated with a random guess.

For the purpose of applying the kinetic rules 20 on the test reaction system 70 (step 80) a new kinetic Monte Carlo simulation is carried out and again a discrete representation of a polymer is obtained (the modified test reaction system 90) after all reactive groups have been reacted. The identical set of descriptors 35 is selected for the modified test reaction system 90 and the corresponding descriptor values 110 are computed (step 100, see Table 1).

The distance 130 in the descriptor space between the two modified reaction systems 40, 90 is then computed (step 120) by using the mean absolute percentage error between the descriptor vectors 60 of the modified reference reaction system 40 and the descriptor vectors 110 of the trial system (modified test reaction system 90).

This distance 130 is then handed over to a Bayesian optimizer (as implemented in a Python library) including the actual composition of the test reaction system 70. Depending on the value of the distance 130 the Bayesian optimizer now decides to adapt the test reaction system 70 (step 140) or to not adapt the test reaction system 70 and propose a recipe (step 160). The decision criterion for the decision to be taken by the Bayesian optimizer is to stop after a maximum of 100 iterations, when no significant change to the distance metric has been recorded anymore. If the Bayesian optimizer decides to adapt the test reaction system 70 an updated test reaction system 180 is obtained by adaption of the test reaction system (step 150) for the next optimization step. The adaption may be effected by the optimizer suggesting new starters/mass fractions based on the optimization history.

The process 70-140 is iterated for approx. 100 iterations/cycles until no change in the distance 130 can be observed anymore or the change lies below a defined threshold. A final set of values of descriptors 110 is obtained with a minimum distance between the descriptors values 60, 110 of the reference/optimized system, i.e. the modified reference and test reaction systems 40, 90:

TABLE 1

| | | | Wiener | | |
|---|---|---|---|---|---|
| System | PD | OHZ | Index | TPSA | fn(OH) |
| Reference recipe | 1.18 | 727 | 11.6 | 71.9 | 2.4 |
| Modified reference reaction | | | | | |
| system 40 | | | | | |
| Proposed recipe | 1.17 | 632 | 11.2 | 70.9 | 2.35 |
| Modified test reaction system | | | | | |
| 90 | | | | | |

As a result the recipe of the test reaction system 70 is proposed (step 160), having the following chemical composition: ethylene oxide (0.54), trimethylolpropane (0.22) and 1,2-propylene glycol (0.24) which is the final test reaction system information 170. This proposed test reaction system 70 is then communicated to a user.

### Example 2

A reference reaction system 10 (the first reaction system) is defined to consists of the following chemical compounds, i.e. recipe: trimethylolpropanol (TMP) (0.5) 1,2-propylene glycol (0.1) and isophthalic acid (0.4) with the composition in mass fractions in parenthesis. As TMP has been labeled as reprotoxic according to REACH, and the polyester reference recipe contains residual amounts of monomeric TMP, the objective of this example is to replace TMP whereas the change of overall polyester characteristics (as given from the descriptor similarity) should be kept at a minimum.

TMP is defined to have three primary alcohol groups, propylene glycol one primary and a secondary alcohol group and isophtalic acid to have two carboxylic acid groups. A kinetic Monte Carlo simulation (as for example in Suderman, Ryan, et al. “Generalizing Gillespie's direct method to enable network-free simulations.” Bulletin of mathematical biology 81.8 (2019): 2822-2848.) is set up where a number of kinetic rules 20 are defined to allow to react the primary alcohol groups with carboxylic acid groups with a relative rate constant of 1, and secondary alcohol groups with a relative rate of 0.1. The kinetic rules 20 are applied on the reference reaction system 10 (step 30) by carrying out the simulation using 100.000 initial seed molecules. A discrete representation of a polymer is obtained (the modified reference reaction system 40), after all reactive groups have been reacted.

Subsequently a number of descriptors 35 is selected and corresponding descriptor values 60 are computed (step 50) from this modified reference reaction system 40 as shown in Table 1: The selected descriptors 35 are: the OH number (OHZ), the average number of OH groups per molecule (fn(OH)), the logarithm of the octanol water partition coefficient (log P) and the Wiener index as obtained from the molecular graph. Explanations regarding the Wiener Index can be found in Todeschini, Roberto, and Viviana Consonni. “Handbook of molecular descriptors”, John Wiley & Sons, 2008. The descriptors values 60 are saved for later usage.

A new test reaction system 70 (second reaction system) is then defined, using starters different from the first reaction system, i.e. the reference system, in the present case, glycerol, di-trimethylolpropanol, diethylene glycole, ethylene glycole, 1,2-propylene glycol and isophthalic acid using the same set of kinetic rules 20 as defined for the reference reaction system 10. This test system is the system to be optimized in the present method. Glycerol is defined to have two primary OH groups and one secondary OH group, di-trimethylolpropanol four primary OH groups, ethylene glycole and diethylene glycole two primary OH groups, 1,2-propylene glycol one primary OH and one secondary group. The composition in mass fractions is initiated with a random guess.

For the purpose of applying the kinetic rules 20 on the test reaction system 70 (step 80) a new kinetic Monte Carlo simulation is carried out and again a discrete representation of a polymer is obtained (the modified second or test reaction system 90) after all reactive groups have been reacted. The identical set of descriptors 35 is selected for the modified test reaction system 90 and the corresponding descriptor values 110 are computed (step 100, see Table 1).

The distance 130 in the descriptor space between the two modified reaction systems 40, 90 is then computed (step 120) by using the mean absolute percentage error between the descriptor vectors 60 of the modified reference system (modified reference reaction system 40) and the descriptor vectors 110 of the trial system (modified second or test reaction system 90).

This distance 130 is then handed over to a Bayesian optimizer (as implemented in a Python library) including the actual composition of the second reaction system 70. Depending on the value of the distance 130 the Bayesian optimizer now decides (step 140) to adapt the test reaction system 70 (step 150) or to not adapt the test reaction system 70 and propose a recipe (step 160). The decision criterion for the decision to be taken by the Bayesian optimizer is to stop after a maximum of 1000 iterations, when no significant change to the distance metric has been recorded anymore. If the Bayesian optimizer decides to adapt the test reaction system 70 an updated test reaction system 180 is obtained by adaption of the test reaction system (step 150) for the next optimization step. The adaption may be effected by the optimizer suggesting new starters/mass fractions based on the optimization history.

The process 70-140 is iterated for approx. 1000 iterations/cycles until no change in the distance 130 can be observed anymore or the change lies below a defined threshold. A final set of values of descriptors 110 is obtained with a minimum distance between the descriptors values 60, 110 of the reference/optimized test system 40, 90, i.e. the first and second modified reaction systems (reference reaction system and test reaction system):

TABLE 1

| System | OHZ | Wiener Index | logP | fn(OH) |
|---|---|---|---|---|
| Reference recipe | 552 | 40 | 0.64 | 3.41 |
| Modified reference reaction | | | | |
| system 40 | | | | |
| Proposed recipe | 538 | 58 | 0.67 | 3.54 |
| Modified test reaction system 90 | | | | |

As a result the recipe of the second reaction system, the test reaction system, is proposed (step 160), having the following chemical composition: di-trimethylolpropane (0.48), 1,2-propylene glycole (0.14), ethyleneglycole (0.04) and isophthalic acid (0.33), which is the final test reaction system information 170. The weight fraction of glycerol was optimized to such a low value, that it was considered not to be necessary for the proposed recipe. This proposed test reaction system 170 is then communicated to a user in order to carry out further lab trials.

1. A computer-implemented method for designing a starting composition of a chemical reaction system for maximizing similarity to a reference reaction system in order to substitute individual components in the reference system, the method comprising:
A) providing information on a reference reaction system, the information comprising components of the reference reaction system and their relative amounts in the reference reaction system;
B) providing information on a test reaction system, the information comprising components of the test reaction system and their relative amounts in the test reaction system;
C) providing at least one first rule for forming and/or cleaving chemical bonds from the components of the reference reaction system and/or species formed from components of the reference reaction system;
D) applying the at least one first rule on the reference reaction system, thereby obtaining a modified reference reaction system;
E) providing at least one descriptor for at least part of the modified reference reaction system and for at least part of a modified test reaction system as recited in step H);
F) determining descriptor value(s) from the at least one descriptor for at least part of the modified reference reaction system;
G) providing at least one second rule for forming and/or cleaving chemical bonds from the components of the test reaction system and/or species formed from components of the test reaction system;
H) applying the at least one second rule on the test reaction system, thereby obtaining a modified test reaction system;
I) determining descriptor value(s) from the at least one descriptor as recited in step E) for at least part of the modified test reaction system;
J) determining the distance and/or the dissimilarity between the descriptor values from steps F) and I) using a measure for the distance in a descriptor space occupied by the at least one descriptor;
K) deciding to adapt or not to adapt the test reaction system depending on the result of the determination of the distance and/or dissimilarity in step J); and
L) if the decision to adapt in step K) has been taken, adapting the test reaction system by changing the components of and/or their relative amounts in the test reaction system yielding an updated test reaction system and subsequently repeating the method starting at step H) based on the updated test reaction system.

2. The method according to claim 1, wherein the components of the reference reaction system as recited in step A) and/or the components of the test reaction system as recited in step B) comprise alkylene oxides, polyfunctional carboxylic acids, carboxylic acid anhydrides, cyclic ethers, carbon dioxide, cyclic carbonates, polyols, polyisocyanates, polyamines, aromatic hydrocarbons, olefins, (meth)acrylates, bisphenols, phosgene, dialkyl carbonates, aldehydes, lactames, glycolides, amino acids, hydroxy-substituted carboxylic acids, or a combination of at least two of the aforementioned components.

3. The method according to claim 1, wherein the at least one first rule recited in C) and/or the at least one second rule recited in G) comprises which functional group of a component is present, which functional group of a component is available for forming a covalent bond or for cleaving a covalent bond, which functional groups of components can react with other functional groups, a threshold criterion when the forming or the cleavage of a covalent bond occurs, or a combination of at least two of the aforementioned rules.

4. The method according to claim 1, wherein the at least one first rule of step C) is identical to the at least one second rule of step G).

5. The method according to claim 1, wherein applying the at least one first rule as recited in step D) and/or applying the at least one second rule as recited in step H) comprises running a kinetic Monte Carlo-simulation, running a molecular Monte Carlo-simulation, running a molecular dynamics simulation, running a Miller-Macosko type calculation, or a combination of at least two of the aforementioned simulations.

6. The method according to claim 1, wherein the modified reference reaction system comprises an ensemble of discrete components and information on the discrete components of the modified reference reaction system and their relative amounts in the modified reference reaction system.

7. The method according to claim 1, wherein the modified test reaction system comprises an ensemble of discrete components and information on the discrete components of the modified test reaction system and their relative amounts in the modified test reaction system.

8. The method according to claim 1, wherein the at least one descriptor as recited in step E) is:
a statistical/compositional descriptor; and/or
a topological descriptor;
a molecular descriptor;
a force field based descriptor;
a quantum chemical descriptor;
a thermodynamic descriptor;
a health, sustainability or environment related descriptor;
or a combination of at least two of the aforementioned descriptors.

9. The method according to claim 1, wherein the distance and/or dissimilarity as recited in step J) comprises the euclidean/L2 distance, the manhattan/L1/cityblock distance, Canberra distance, Chebyshev distance, Mahalanobis distance, Minkowski distance, Rogers-Tanimoto dissimilarity, Russell-Rao dissimilarity, Sokal-Michener dissimilarity, Sokal-Sneath, mean absolute percentage error, Yule dissimilarity, cosine dissimilarity, dice dissimilarity, or a combination of at least one of the aforementioned distances and/or dissimilarities.

10. The method according to claim 1, wherein the decision as recited in step K) comprises one or more of the following a decision criteria:
a threshold value for the distance and/or dissimilarity in step J),
a threshold value for the change in distance and/or dissimilarity relative to one or more previously executed steps J),
a threshold value for the execution time for the method,
a threshold value for the number of repetitions of the method, and
a threshold value for at least one of the descriptors.

11. The method according to claim 1, wherein adapting the test reaction system by changing the components of the test reaction system and/or their relative amounts in the test reaction system as recited in step L) comprises executing an algorithm selected from: random search, grid search, Bayesian optimization, simplex optimization, evolutionary optimization, genetic algorithm, particle swarm optimization, Metropolis-Hastings Markov-chain Monte-Carlo, adaptive Markov-chain Monte-Carlo, simulated annealing, parallel tempering, mixed linear and non-linear programming, or a combination of at least two of the aforementioned algorithms.

12. The method according to claim 1, further comprising communicating a final test reaction system to a user if the decision has been taken in step K) not to adapt the test reaction system in step K).

13. A data processing apparatus comprising means for carrying out the method of claim 1.

14. A computer program product comprising instructions which, when the program is executed by a computer, cause the computer to carry out the method of claim 1.

15. A computer-readable storage medium comprising instructions which, when executed by a computer, cause the computer to carry out the method of claim 1.

16. The method of claim 6, wherein determining descriptor value(s) from the at least one descriptor for at least part of the modified reference reaction system in step F) is carried out using atomistic resolution information for each discrete component.

17. The method of claim 7, wherein determining descriptor value(s) from the at least one descriptor for at least part of the modified test reaction system in step I) is carried out using atomistic resolution information for each discrete component.

18. The method according to claim 8, wherein:
the statistical/compositional descriptor comprises at least one descriptor selected from the group consisting of number average molecular weight, weight average molecular weight, viscosity average molecular weight, polydispersity index, mean functionality, OH number, and acid number;
the topological descriptor comprises at least one descriptor selected from the group consisting of Wiener Index, Randic connectivity index, Balaban-J index, Ipc index, Zagreb index, Bertz index, chi molecular connectivity indices, Kier-Hall valence connectivity index, seniority and priority indices, kappa shape indices, BCUT indices, Estate indices, Walk and path counts, and extended connectivity fingerprints;
the molecular descriptor comprises at least one descriptor selected from the group consisting of Labute's approximate surface area, solvent-accessible surface area, radius of gyration, 2D autocorrelations, Eigenvalue-based descriptors, RDF descriptors, 3D-MORSE descriptors, WHIM descriptors, GETAWAY descriptors, functional group counts, number of rotatable bonds, number of hydrogen acceptors and donors, Gasteiger/Marsili Partial Charges, topological polar surface area, and molar refractivity;
the force field based descriptor comprises at least one descriptor selected from the group consisting of intramolecular potential energy, radial distribution function, bond angle distribution function, and dihedral distribution functions;
the quantum chemical descriptor comprises at least one descriptor selected from the group consisting of atomic charges, HOMO and LUMO energies, molecular hardness, molecular polarizability, dipole moment, total energy, and molecular quantum number descriptor;
the thermodynamic descriptor comprises at least one descriptor selected from the group consisting of octanol-water partition coefficient, sigma-profile, sigma moment, polarity moment vector, activity coefficient, chemical potential, free energy, octanol-water partition coefficient, and solubility; and/or
the health, sustainability or environment related descriptor comprises at least one descriptor selected from the group consisting of toxicity, carbon dioxide equivalents, carbon footprint, energy consumption, product recycling rate, sustainability index, supply chain miles, water footprint, and price.
